(19) United States
(12) Patent Application Publication
(10) Pub. No.: US 2008/0077010 A1
(43) Pub. Date: Mar. 27, 2008

(54) Method and Aparatus for Presenting Information Concerning Flow Behavior of a Bodyfluid Externally Measured by Ultrasound

(75) Inventors: Eric Cohen-Solal, Ossining, NY (US); Shervin Ayati, Carlisle, MA (US); Balasundara Iyyavu Raju, Tarrytown, NY (US)

Correspondence Address: PHILIPS INTELLECTUAL PROPERTY & STANDARDS, P.O. BOX 3001, BRIARCLIFF MANOR, NY, 10510, US

(73) Assignee: Koninklijke Philips electronics N.V., Eindhoven (NL)

(21) Appl. No.: 11/576,113

(22) PCT Filed: Sep. 22, 2005
(86) PCT No.: PCT/IB05/53129

§ 371 Date: Mar. 27, 2007

Related U.S. Application Data
(60) Provisional application No. 60/613,996, filed on Sep. 28, 2004.

(51) Int. Cl. A61B 8/00

(52) U.S. Cl. 600/441

(57) ABSTRACT

A flow behavior monitor for presenting ultrasound measurements of an indicia of flow behavior of a fluid in a subject. The indicia of flow behavior are calculated for several frequency slices within a Doppler signal power spectrum and these indicia may be used to determine pulsatility and/or blood flow, as well as other parameters of flow behavior. Because of the robust nature of the calculated indicia, the flow behavior monitor has particular use in an Automated or Semi-Automated External Defibrillator (AED) for determining whether to defibrillate a patient

The present invention relates generally to the field of medical ultrasound diagnostics and, more specifically, to a method and apparatus for presenting information concerning the flow behavior of a body fluid using an externally attached ultrasound device.

Patient monitoring systems are used to provide real-time assessment of vital clinical parameters of the patient in emergencies and during operative procedures, post-operative intensive care, and other life-threatening situations. Typically, these systems display vital sign measurements, such as ECG (Electrocardiogram), EEG (Electroencephalogram), SPO₂ (pulse oximetry), CO₂ (blood level of carbon dioxide), blood pressure, etc. However, the immediate determination of the condition of the heart and the flow of blood through the patient's body is often key to the caregiver's diagnosis and actions regarding the patient. As such, the monitoring of the heart condition and the flow behavior of the blood becomes critical in the operating room during surgery, in the intensive care unit when an alarm occurs, when an emergency medical technician (EMT) is providing care to an unconscious person, or when the exact nature of the malady is not known.

Thus, in emergencies and during operative procedures, the assessment of the pulse state of the patient is essential for both diagnosis of the problem and determining the appropriate therapy for the problem. In emergency situations or any situation where the full-scale monitoring equipment of a hospital or clinic is not available, the presence of a cardiac pulse in a patient is typically detected by palpating the patient's neck and sensing palpable pressure changes due to the change in the patient's carotid artery volume. When the heart's left ventricle contracts during a heartbeat, a pressure wave is sent throughout the patient's peripheral circulation system. A carotid pulse waveform rises with the ventricular ejection of blood at systole and peaks when the pressure wave from the heart reaches a maximum. The carotid pulse falls off again as the pressure subsides toward the end of the pulse.

The absence of a detectable cardiac pulse in a patient is a strong indicator of cardiac arrest. Cardiac arrest is a life-threatening medical condition in which the patient's heart fails to provide blood flow to support life. During cardiac arrest, the electrical activity of the heart may be disorganized (ventricular fibrillation), too rapid (ventricular tachycardia), absent (asystole), or organized at a normal or slow heart rate without producing blood flow (pulseless electrical activity).

The form of therapy to be provided to a patient in cardiac arrest depends, in part, on an assessment of the patient's cardiac condition. For example, a caregiver may apply a defibrillation shock to a patient experiencing ventricular fibrillation (VF) or ventricular tachycardia (VT) to stop the unsynchronized or rapid electrical activity and allow a perfusing rhythm to return. External defibrillation, in particular, is provided by applying a strong electric shock to the patient's heart through electrodes placed on the surface of the patient's body. If the patient lacks a detectable pulse and is experiencing asystole or pulseless electrical activity (PEA), defibrillation cannot be applied and the caregiver may perform cardiopulmonary resuscitation (CPR), which causes some blood to flow in the patient.

Before providing therapy such as defibrillation or CPR to a patient, a caregiver must first confirm that the patient is in cardiac arrest. In general, external defibrillation is suitable only for patients that are unconscious, apneic, pulseless, and in VF or VT. Medical guidelines indicate that the presence or absence of a cardiac pulse in a patient should be determined within 10 seconds. For example, the American Heart Association protocol for cardiopulmonary resuscitation (CPR) requires a healthcare professional to assess the patient's pulse for five to ten seconds. Lack of a pulse is an indication for the commencement of external chest compressions. Assessing the pulse, while seemingly simple on a conscious adult, is the most often failed component of a basic life support assessment sequence, which may be attributed to a variety of reasons, such as lack of experience, poor landmarks, or a bias to either finding or not finding a pulse. Failure to accurately detect the presence or absence of the pulse will lead to adverse treatment of the patient either when providing or not providing CPR or defibrillation therapy to the patient.

Electrocardiogram (ECG) signals are normally used to determine whether or not a defibrillating shock should be applied. However, certain rhythms that a rescuer is likely to encounter cannot be determined solely by the ECG signal, e.g. pulseless electrical activity; diagnoses of these rhythms require supporting evidence of a lack of perfusion (i.e., blood flow) despite the myocardial electrical activity as indicated by the ECG signal.

Because the pulse check or blood flow measurement is performed manually, it is subject to human error, and in an emergency situation where time is of the essence, the amount of time taken for the manual pulse state assessment is too long thereby causing detrimental results. A reliable pulse state assessment device is needed to solve these limitations.

Even when the ECG analysis is performed, it is possible that the results may mislead the rescuer into taking the wrong course of action. For instance, after a myocardial infarction (MI), the patient may enter a state of pulseless electrical activity (PEA) where the ECG will register normal electrical activity, but there is no pulse present. Because the ECG analysis shows a normal rhythm, the rescuer will misinterpret the data as showing a “pulse”, and the rescuer would take no further action, thereby gravely endangering the patient. Conversely, if a rescuer incorrectly concludes that the patient has no pulse (because of a necessarily rushed preliminary evaluation or false determination of PEA), and proceeds to provide therapy, such as CPR, the patient's chance for recovery of circulation is curtailed.

Thus, in order for a rescuer to quickly determine whether or not to provide therapy to a patient, it is necessary to develop an integrated system that is quickly and easily able to analyze the patient's pulse, the amount of blood flow, and perhaps the ECG signals in order to correctly determine whether there is any pulsatile flow in the arteries of the patient. This necessity is particularly dire in situations or systems. in which the rescuer is untrained and/or inexperienced person, as is the case in the system described in U.S. Pat. No. 6,575,914 to Rock et al., which is assigned to the same assignee as the present invention, is hereby incorporated by reference in its entirety, and will be referred to hereafter as “the Rock patent”. The Rock patent discloses an Automated External Defibrillator (AED) (hereinafter, AEDs or Semi-Automated External Defibrillators—SAEDs—will be jointly referred to as AEDs) which can be used by first-responding caregivers with little or no medical training to determine whether or not to apply defibrillation to an unconscious patient.

The Rock AED has a defibrillator, a sensor pad for transmitting and receiving Doppler ultrasound signals, two sensor pads for obtaining an ECG signal, and a processor which receives and assesses the Doppler and ECG signals in order to determine whether defibrillation is appropriate for the patient (i.e., whether or not there is a pulse). The Doppler pad is adhesively secured to a patient's skin above the carotid artery to sense the carotid pulse (which is a key indicator of sufficient blood pulsatile flow).

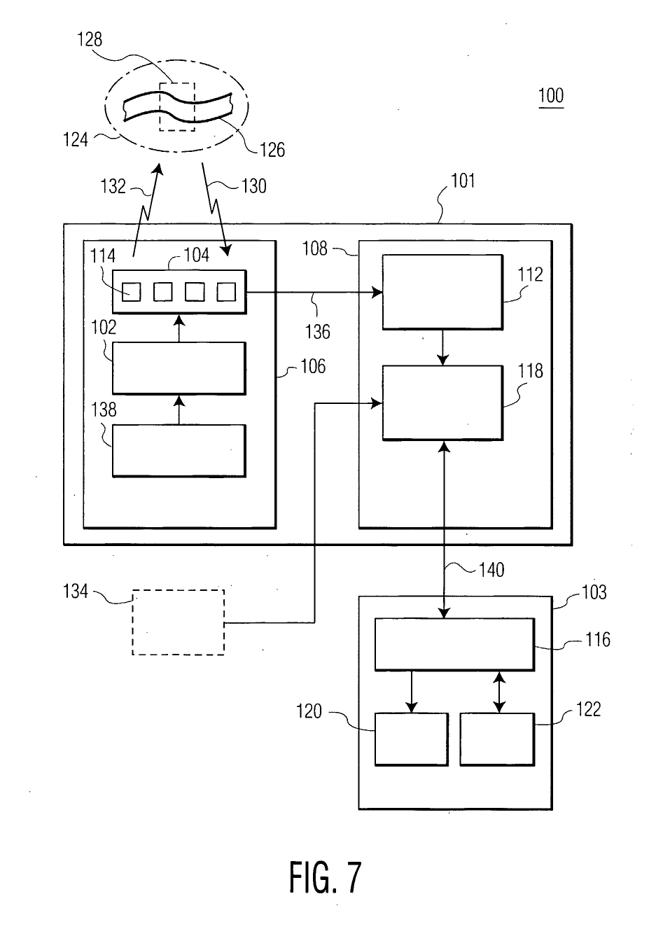

Specifically, the processor in the Rock AED analyzes the Doppler signals to determine whether there is a detectable pulse and the analyzes the ECG signals to determine whether there is a “shockable rhythm” (see, e.g., FIG. 7 and accompanying description at col. 6, line 60, to col. 7, line 52, in the Rock patent). Based on the results of these two separate analyses, the processor determines whether to advise defibrillation or not (id.). Although the Rock patent discusses “integrating” the Doppler and ECG signals, the processor in the Rock AED merely considers the results of both analyses and does not integrate, either mathematically or analytically, the Doppler and ECG signal analyses.

The determination of a detectable pulse by the processor in the Rock AED is made by comparing the received Doppler signals against “a threshold statistically appropriate with the Doppler signals received” (col. 7, lines 13-14, the Rock patent). However, there is at least one problem with using such a threshold analysis of the Doppler signals: the wide variety of body shapes and sizes, steady state (i.e., healthy) blood flows, steady state blood pressures, etc. in humankind. Because an AED may be located anywhere that untrained rescuers could operate such a device (e.g., an airplane, a train, a bus, a lobby in a large building, an infirmary, etc.), and the pads of an AED may be placed on a man, a woman, a child, a full-grown adult, an elderly person, someone with a naturally low pulsatile flow, etc., it is difficult, if not impossible, to determine a “universal” threshold that can adequately cover the variety of humans which may or may not need cardiac resuscitation.

Moreover, even in an AED where multiple transducers are used to ensure that one of them captures the artery, the best transducer in a multi-transducer pad might still be offset from the artery by an unknown distance, which means the signals are different compared to the no offset case.

Thus, there is a need for a method and apparatus which can adequately assess and present information concerning the flow behavior of the blood of an individual without a priori measurements or knowledge of that particular individual. Furthermore, there is a need for a method and apparatus which can inform an inexperienced and/or untrained user of an AED or any other defibrillation device whether there it is appropriate to defibrillate a patient.

A flow behavior monitor according to the present invention presents at least one of visual and audio output representing at least one measurement of an indicia of flow behavior of a fluid within a subject. The indicia of the flow behavior of the body fluid is determined by first calculating a Doppler power spectrogram from ultrasound signals backscattered from a body fluid (such as blood in the carotid artery), and then calculating the power spectra of individual frequency slices within the calculated Doppler power spectrogram are then calculated. The indicia are determined from the power spectrum of each individual frequency slice. Flow behavior may refer to the state of blood perfusion, the state of pulse, the heart beat rate, the flow activity of the blood, and/or the pulsatile activity of the blood. It is contemplated that the present invention may be used on other bodily fluids, as well as other colloidal or emulsion solutions contained in inanimate objects.

In comparison with the prior art, in which the Doppler signal is analyzed over the entire frequency spectrum, the flow behavior monitor according to the present invention uses a flow behavior indicia which isolates and analyzes individual frequency bands, thereby recognizing the signal of a weak flow in an individual frequency band, rather than allowing such a signal to be lost in the background noise if the entire frequency spectrum is used. In other words, the signal is better revealed compared to the noise if a small relevant frequency band is used rather than the entire spectrum.

The flow behavior monitor is also an apparatus for medical ultrasound diagnostics and monitoring a patient that provides medical staff with information related to mechanical activity of the patient's heart.

In a first aspect of the present invention there is provided an apparatus for medical ultrasound diagnostics and monitoring a patient that comprises at least one ultrasonic transducer, a data processor, and an operator interface module. The data processor measures and/or detects at least one indicia of flow behavior using calculations performed in frequency bands where, during a cardiac cycle, the power of the Doppler signal has a maximal signal-to-noise ratio and/or maximal variations. As indicated above, flow behavior may include the perfusion in a blood vessel (e.g., carotid artery), the heart beat rate, and the pattern of pulsatile activity of the heart. In one embodiment, the operator interface module includes at least one display presenting the measurements of the at least one indicia of flow behavior and at least one optional source of an audible signal presenting a portion of the diagnostic information relating to an audible pattern of the heart beat rate.

In a second aspect of the present invention there is provided a defibrillation system, comprising the inventive apparatus for ultrasound diagnostics and monitoring a patient, a defibrillating unit having a controlled high-voltage source, and a controller of the defibrillating unit. In one application, the apparatus is used to diagnose a patient, provide information for determining whether to defibrillate the patient, and monitor the patient's post-treatment conditions.

Other objects and features of the present invention will become apparent from the following detailed description considered in conjunction with the accompanying drawings. Although fundamental novel features of the present invention as applied to the preferred embodiments shown and described below are pointed out, it will be understood that various omissions and substitutions and changes in the form and details of the embodiments described and illustrated, and in their operation, and of the methods described may be made by those skilled in the art without departing from the spirit of the present invention. It is the intention that the present invention be limited only as indicated by the scope of the claims appended hereto.

The teachings of the present invention will become apparent by considering the following detailed description in conjunction with the accompanying drawings, in which:

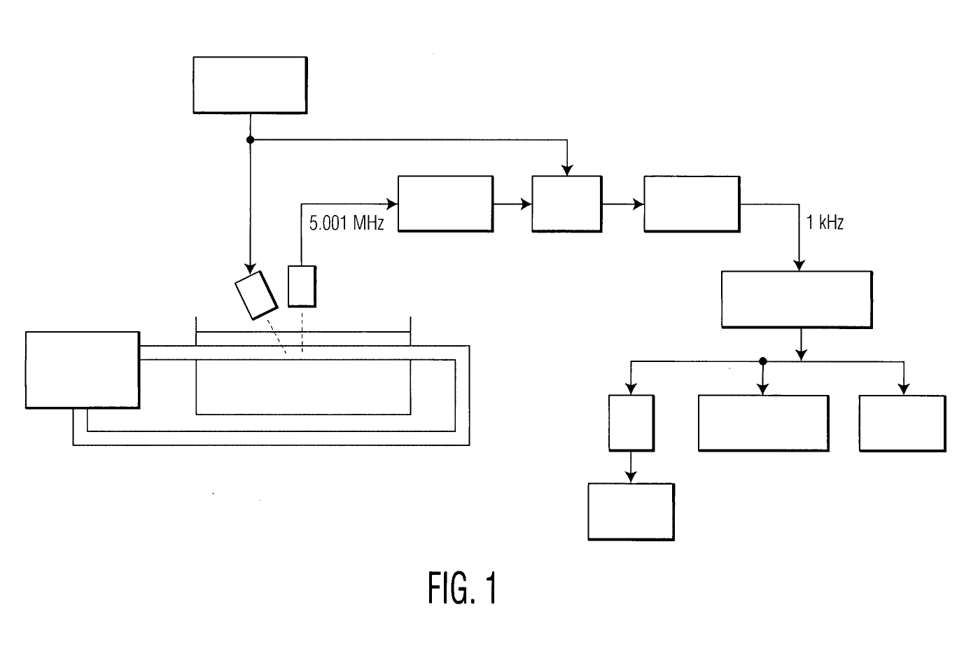

FIG. 1 shows a schematic of an experimental set-up used to test the feasibility of a method and apparatus according to the present invention;

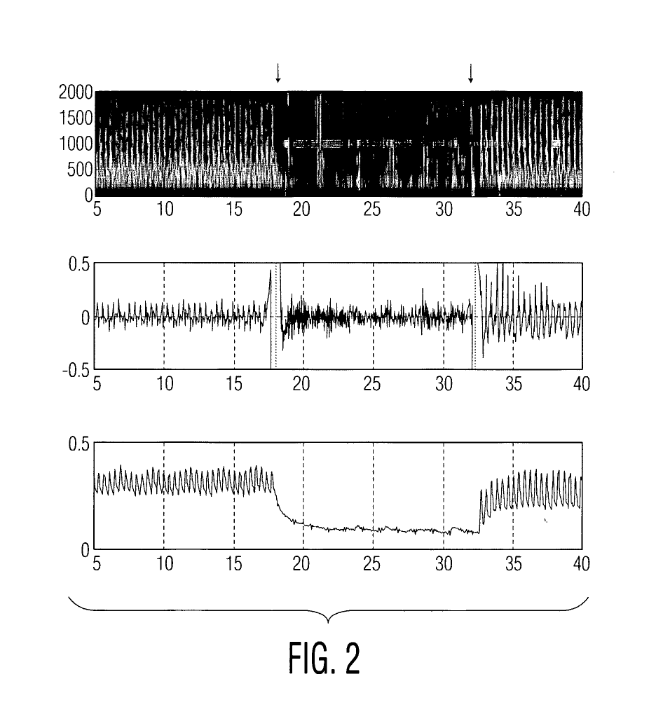

FIG. 2 shows a Doppler spectrogram with the corresponding ECG and arterial blood pressure (ABP) signals taken of a heart in VF using the experimental set-up of FIG. 1;

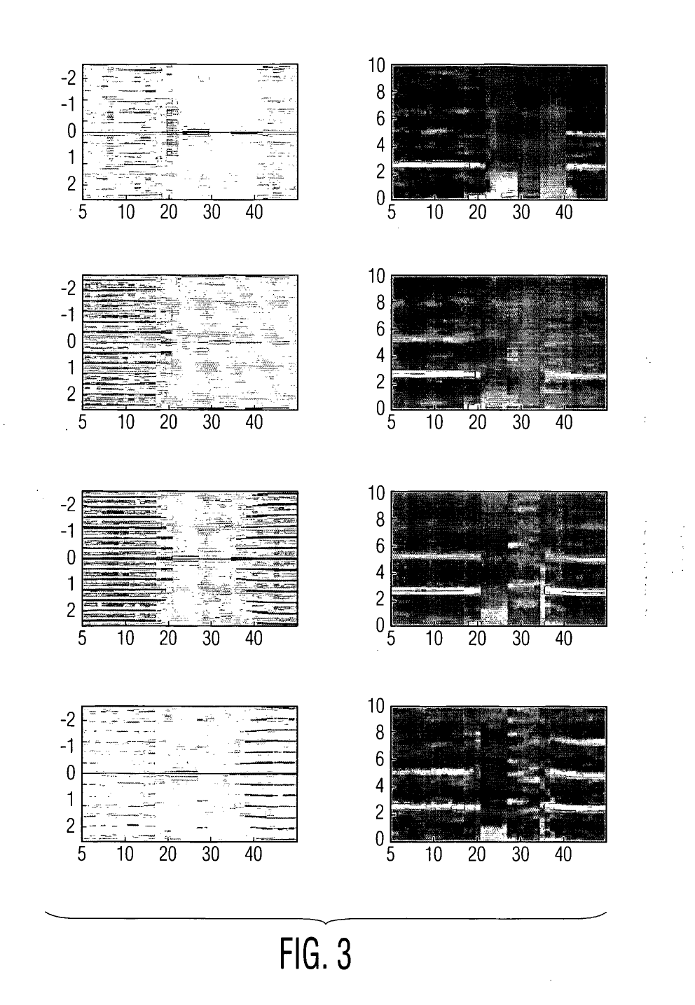

FIG. 3 shows the auto-correlation, and the Fourier Transform of the auto-correlation, of four frequency slices from the Doppler spectrogram of FIG. 2, according to a preferred embodiment of the present invention;

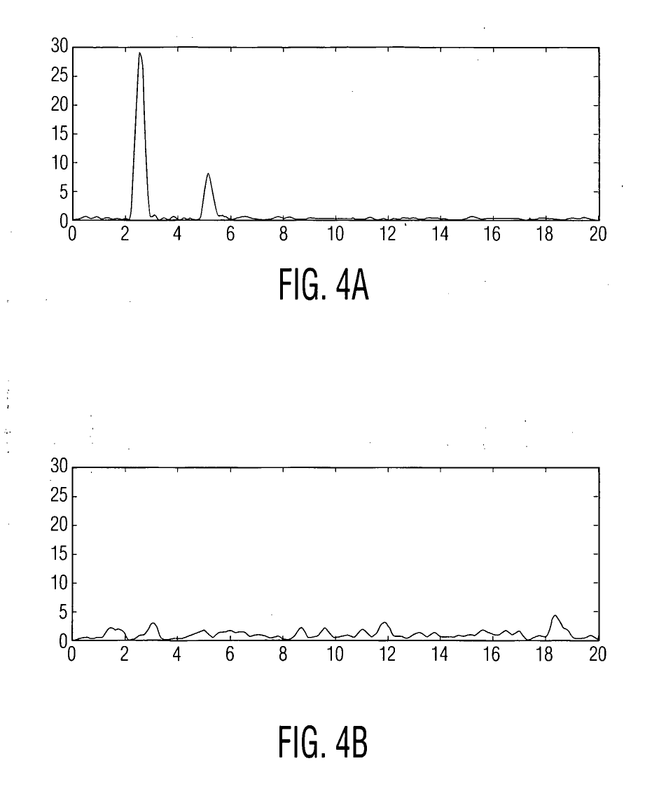

FIG. 4 shows the Fourier Transforms at 10 seconds and at 30 seconds of the auto-correlation of the 1150-1350 Hz frequency slice from FIG. 3, according to a preferred embodiment of the present invention;

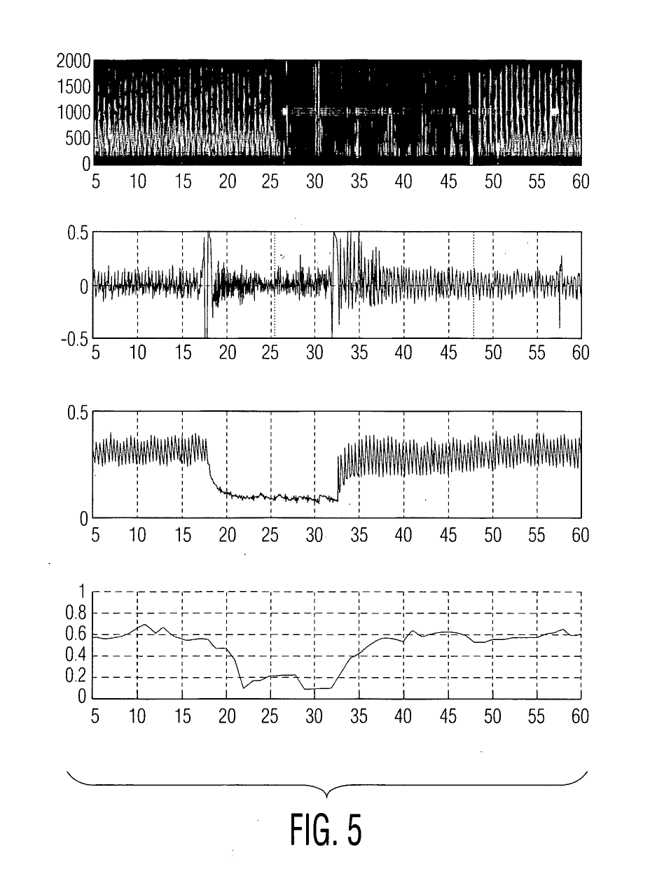

FIG. 5 shows, in the bottom graph, the third pulsation index calculated from the data in FIG. 2, which is shown in the top three graphs, according to a preferred embodiment of the present invention;

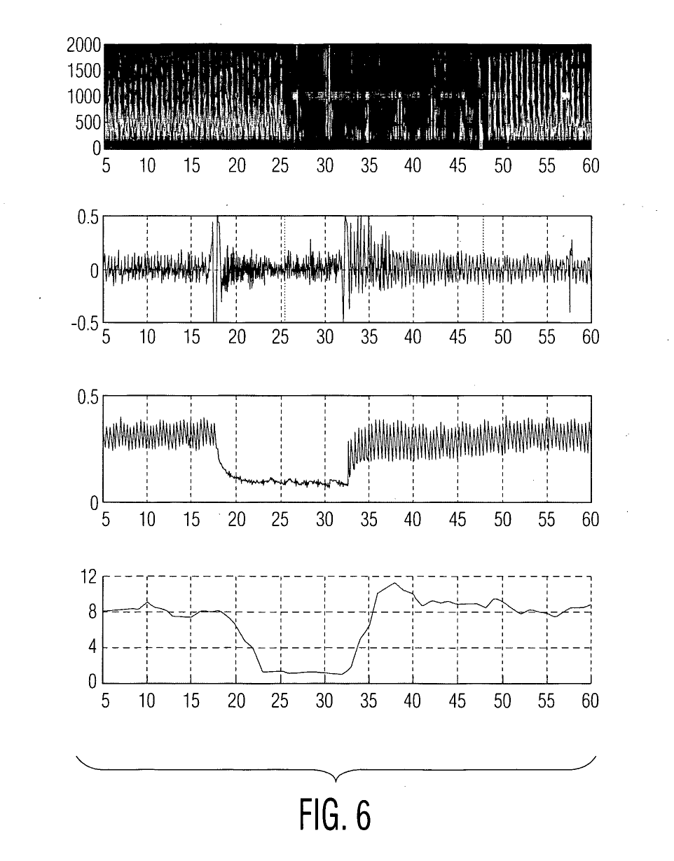

FIG. 6 shows, in the bottom graph, the flow index calculated from the data in FIG. 2, which is shown in the top three graphs, according to a preferred embodiment of the present invention;

FIG. 7 depicts a block diagram of an exemplary apparatus of the kind that may be used for ultrasound medical diagnostics and monitoring a patient in accordance with one embodiment of the present invention;

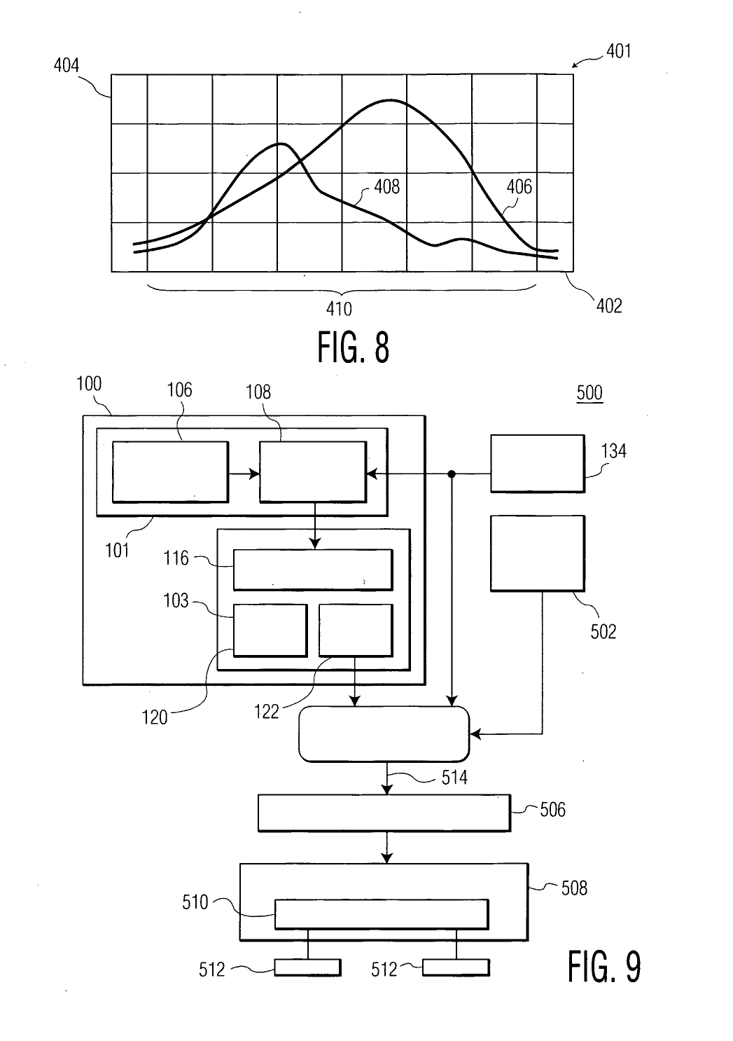

FIG. 8 depicts an exemplary diagram illustrating displaying in the apparatus of FIG. 7 the spectral power distribution of a Doppler signal data during a cardiac cycle; and

FIG. 9 depicts a block diagram of an exemplary defibrillating system having the apparatus of FIG. 1 in accordance with one embodiment of the present invention.

Herein, identical reference numerals are used, where possible, to designate identical elements that are common to the figures. The images in the drawings are conventionally simplified for illustrative purposes and are not depicted to scale.

The appended drawings illustrate exemplary embodiments of the invention and, as such, should not be considered limiting the scope of the invention that may admit to other equally effective embodiments.

This detailed description is broken into two sections: the first section describes a novel and inventive measurement of flow behavior, while the second section describes the apparatus for presenting the measurement results to a caregiver.

As discussed above, assessing the pulse state of a patient represents a challenging task, especially in emergencies and during operative procedures, post-operative intensive care, and other life-threatening situations. In such situations, while detecting electrical activity of the heart, an electrocardiogram (ECG) may inadvertently mask the lack of the mechanical activity (i.e., blood pumping functionality) of the heart, thus providing inadequate diagnostic data (leading the caregiver to conclude that there is a pulse) when the heart is in the state of pulseless electrical activity (PEA).

Analyzing the pulsing activity of the heart is problematic if there is weak perfusion, because of the difficulties associated with resolving small variations of a mean (or central) Doppler frequency of the echo signal (i.e., Doppler frequency shifts) at high levels of background spectral noise. Such limitations have a negative impact on the capabilities and clinical efficiency of medical systems using ultrasonic diagnostic information. This is particularly the case when the medical system is intended for use by laymen, such as programmable defibrillators (AED).

The preferred embodiments of the present invention use selective calculations of the power spectrum in each of a plurality of frequency bands of the Doppler spectrogram. The plural frequency bands or slices may comprise the entire frequency spectrum of the Doppler spectrogram, or only two or more preselected slices within the spectrum. In one embodiment, the preselected slices are selected so that their combination will adequately cover as many of the possible indicators of flow behavior in the largest variety of humans (or other subjects). The frequency slices may be of equal or unequal size. Furthermore, the size and location of the frequency slices may be dynamic, i.e., the size and/or location of the frequency slices may change during the analysis of a particular patient.

Any method of ultrasound Doppler can be used with the present invention. The simplest approach is the continuous-wave (CW) Doppler method. In this method, one ultrasound transducer emits a continuous wave signal and another transducer receives the backscattered signal from the region of overlap between the two beams. The received signal, after suitable amplification, is sent to a mixer where signals at the sum and difference frequencies are produced. A low pass filter removes the sum frequency leaving the low frequency base band signal that has a frequency equal to the Doppler frequency. This CW method determines the classical Doppler frequency shift. The drawback of this method is that there is no localization of the signal from blood since the signals from all tissue locations in addition to signals from blood are intrinsically combined.

An alternate method is the pulsed-wave (PW) Doppler technique. In this method, the classical frequency shift is not used. Rather, the phase of the base band signal after demodulation and its change over a repeated set of acquisitions is utilized in reconstructing the Doppler signal. In this method it is possible to select the exact depth at which to analyze the blood or tissue motion. The drawback of this approach is that the electronics required is more complex than the CW case. Also there is the possibility of aliasing if the pulse repetition frequency is not higher than twice the expected Doppler frequency shift. In yet another method, commonly referred to as the Color Doppler technique, the motion of scatterers is determined through a correlation approach. Reflected signals from repeated insonifications are analyzed in order to determine an average motion of scatterers.

Although these approaches are mentioned here, any other Doppler method could be used with the present invention, as would be understood by one skilled in the art.

In experiments studying the feasibility of a method and system according to the present invention, the simpler CW method was used. In the preferred embodiment, it is not necessary to know precisely from where the signals were reflected. The backscattered signals are obtained from both the blood flow and all other tissues up to a depth limited by the attenuation of the signal. In order to separate the blood flow from tissue motion, a high pass wall filter was used, based on the assumption that the tissue velocities are of much lower frequency than that of blood flow. The experiments were performed on pigs because their cardiovascular systems are similar to that of humans.

FIG. 1 shows a schematic of the CW experimental set-up, in which a single element transducer (Panametrics, Waltham, Mass.; Model A309S) is excited by an arbitrary waveform generator (Wavetek/Fluke, Everett, Wash.; Model 295), and another transducer identical to the transmit transducer collects the Doppler shifted backscattered echoes. The received signal is amplified using two low noise pre-amplifiers (Minicircuits, Brooklyn, N.Y.; Model ZFL-500LN) each having at least 24 dB of gain, a low noise figure of 2.9 dB, and a rated power output capacity of 5 dBm at 1 dB compression point. The signal after pre-amplification is sent to a mixer (Minicircuits; Model ZP-3MH or other suitable mixers). The mixer also receives a part of the excitation signal from the Wavetek generator at its local-oscillator port. The output of the mixer contains a signal that is the sum and difference of the excitation signal and the received signal. A low pass filter (Minicircuits; Model BLP-1.9) removes the signal at the sum frequency leaving the Doppler signal at the difference frequency to pass through.

Three signals were simultaneously recorded: Ultrasound Doppler, ECG, and Arterial Blood Pressure (ABP). Since it was not a priori possible to estimate the level of Doppler signal from pigs, several additional mixers, filters, and attenuators were made available to allow for flexibility in recording the signals. Filtering (including wall filtering) and amplification of the Doppler signals was performed using another system from Krohn-Hite Corporation (Brockton, Mass.). The Krohn-Hite system was a two-channel tunable filter and amplifier (Model 3382) with a tunable frequency range between 0.1 Hz to 200 kHz. This system had a very sharp cut off frequency (48 dB/octave) which was preferred for the Doppler wall filtering. It also offered considerable flexibility in selecting the gain and filter settings. Each of the channels had a pre-filter gain stage with up to 50 dB gain in 10 dB steps, and a post-filter stage with gain up to 20 dB in 0.1 dB steps. The cut-off frequency could be specified with a resolution of 3 digits. One of the channels in this instrument was used for the high-pass wall filtering and the other for low pass filtering to reduce noise. The high pass cut-off was initially set at 50 Hz but changed to 200 Hz for later experiments. The low pass cut-off was set to 3 kHz.

The Doppler spectrogram created using the data recorded during a typical experiment is shown in FIG. 2. The Doppler spectrogram is essentially a Short Time Fourier Transform (FT) of the Doppler signal and is similar to those displayed on commercial high-end ultrasound systems. Beneath the Doppler spectrogram are shown the corresponding ECG and the ABP signals. The temporal and −3 dB frequency resolutions of the spectrogram were 25 ms and 160 Hz respectively.

FIG. 2 describes the different phases of the cardiac activity during a typical experiment. At the start of the experiment, the heart has its normal beating state. The ECG shows a normal beating rhythm, and the ABP shows the pulsatile nature of the blood pressure in the carotid artery. The corresponding Doppler spectrogram also shows the pulsatile behavior in that the Doppler power moves from the higher frequencies during the systolic phase to the lower frequencies during the diastolic phase. The period of the Doppler spectrogram corresponds to the period of the ABP. At about 18 seconds, an electrical shock is applied to the open heart, which puts the heart in a state of VF. At this point, the ECG loses its normal rhythm and the ABP drops drastically. The corresponding Doppler spectrogram does not show the normal pulsatile behavior seen before the VF. After the animal is in VF for about 15 seconds, a defibrillation shock is applied, causing the heart to recover its beating activity. The ECG returns to the normal rhythm and the ABP increases to a normal rate. The Doppler spectrogram returns to its normal pulsatile state. Although the spectrogram lost its normal pulsatile signature during the period of VF, some activity of the heart, especially at the low Doppler frequencies, could be seen. When the Doppler signal is played on an audio speaker, the pulsatile nature during the initial and the recovery states is apparent, as is the loss of pulsatility during the VF state.

Having created a set of measurements from a series of experiments like that shown in FIG. 2 conducted using the experimental set-up of FIG. 1, various indicia of flow behavior were examined.

As discussed above, in the present invention, the Doppler spectrogram is broken down into two or more frequency slices (i.e., a slice being taken horizontally across the spectrogram shown in FIG. 2) because it is easier to detect pulsatility within a specific frequency band rather than across the total Doppler power spectrum across all frequencies. The specific band in which a pulsatile flow may become apparent depends on many factors, such as the strength of the flow, the Doppler angle, the size of the patient, the normal pulsatile flow of the patient, etc.

In the experiments, four frequency bands were selected for analysis: 225 to 425 Hz, 650 to 850 Hz, 1150 to 1350 Hz, and 1650 to 1850 Hz. These frequency bands were chosen so as to avoid unexpected electrical noise in the recording unit that mostly occurred at 1 kHz, and sometimes at 500 and 1500 Hz. The total Doppler power in these frequency bands was computed as a function of time, which, as mentioned above, is essentially the same as taking a horizontal slice through the spectrogram in FIG. 2. Once the Doppler power within each of the specific frequency bands was calculated, the unbiased auto-correlation of the Doppler power was computed within a 5-second window, which can be seen on the left-hand side of FIG. 3. The time period of 5 seconds corresponds to several cardiac cycles, and is a good trade-off between allowing sufficient time for periodicity estimation and making this period short enough to evaluate as quickly as possible. The auto-correlation function has the property of clearly exposing any periodicity in the signal. The auto-correlation was normalized to have values between −1 and +1. The window was progressively advanced in time (a sliding window) so as to obtain the auto-correlation for the duration of the experiment. The Fourier Transform (FT) of the auto correlation, referred to as the power spectrum, was also computed, and is shown on the right-hand side of FIG. 3. It is expected that during pulsatile activity, the power spectrum would contain a peak at a frequency corresponding to the period of the pulsatile activity. For instance, if the heart rate were 60 beats per minute, the power spectrum would show a peak at a frequency of 1 Hz.

The pulsatile nature of the Doppler power spectrum during the initial and recovery states is readily apparent in the auto correlations shown in FIG. 3. The power spectra during these periods show a peak corresponding to the period of the auto-correlation. It can also be seen that some of the frequency bands (e.g., 1150 to 1350 Hz) expose the periodic nature better than the others.

FIG. 4 shows power spectra in the 1150 to 1350 Hz band obtained from FIG. 3 at two specific time instants. The two time instants correspond to the cases when the 5 sec windows used in the auto correlation ended at 10 and 30 seconds respectively. The former corresponded to the initial state of the heart before fibrillation and the latter to the VF state. It can be seen that during the initial state, the FT showed a peak at a frequency of about 2.58 Hz, which corresponded to a heart rate of 155 beats per minute, the same as that measured by the defibrillator monitoring the ECG signal. In this particular case, a significant second harmonic is also seen at twice the fundamental frequency. During the VF state however the FTs do not show the presence of a strong peak.

It should be noted that the term frequency is used herein differently in different contexts: ultrasound frequency is in the MHz range, the Doppler frequency is in the hundreds of Hz to kHz range, and finally pulse frequency corresponding to the pulsatility of the flow is usually in the range of a few Hz. The different usages should be apparent to one skilled in the art from the context.

The first proposed indicia for flow behavior is directed to measuring the pulsatility of the flow by the periodicity of the Doppler signal. This indicia, called the “pulsation index”, is a ratio of the power in a peak in the power spectrum of a frequency slice (e.g., FIG. 4) to the power in the total power of the power spectrum of the frequency slice (or just the background of the total power spectrum, i.e., the spectrum excluding the peak or peaks).

When finding the pulsation index according to a preferred embodiment of the present invention, the Doppler power in several frequency bands is computed as a function of time, followed by the computation of the auto-correlations and power spectra, as has been described above. A peak-searching algorithm then determines the frequency at which the power spectrum is a maximum. The fraction of the total power contained within a narrow band around this frequency peak is determined. For the case of normal pulsatile flow, one would expect that a significant portion of the total power would be present in this narrow band whereas that would not be the case when pulsatile flow is absent.

A priori assumptions based on physiology could be used to restrict the search space for the location of the peak in the power spectrum. For instance, for the data recorded from pigs, it could be assumed that during normal flow in the carotid, the heart rate would be between 40 and 240 beats per minute. Thus the algorithm would search for the global peak between 0.67 and 4 Hz. The bandwidth of the narrow band is determined by the total time duration of the auto-correlation. Since the auto-correlation was computed over a lag time of T=5 seconds, the useful bandwidth was taken to be 80% of 4/T=0.64 Hz (80% would capture most of the main lobe width). There are a few cases where no maximum were to be found within this range. In such cases, the algorithm would set the computed index to be zero.

Although many possible pulsation indices are possible in accordance with the present invention, three possible pulsation indices will be considered herein. In each case, the pulsation index takes values ranging between 0 and 1, with higher values expected for the flow case and lower values for the no flow case.

The first pulsation index is the ratio of the power in the narrow band around the frequency peak to the total power in the signal over all the frequencies.

The second pulsation index is the ratio of the sum of total power in the narrow bands around the peak frequency and at twice the peak frequency (referred to as the second harmonic frequency) to the total power in all frequencies. This measure accounts for the fact that the pulsatile signal is not sinusoidally periodic, and consequently can contain additional harmonics. For simplicity, only the second harmonic is included and the higher order harmonics are not considered.

The third pulsation index is the ratio of the power in the narrow band around the peak frequency to that of the total power excluding the second harmonic. This is similar to the first measure except that the denominator excludes the power in the second harmonic.

While all three indices quantify the periodic behavior in the Doppler power, a heuristic analysis can be invoked to prefer one over the other two. In this analysis, it is assumed that the flow case contains a peak at a fundamental frequency and a smaller peak at the second harmonic, whereas the no flow case is essentially noise for which the power spectrum is essentially low and constant at all frequencies.

For the no flow case, the second pulsation index would be about twice that of the first pulsation index, since twice the amount of noise is present in the numerator. For the flow case, the second pulsation index would be less than twice that of the first pulsation index, since the second harmonic is of smaller magnitude than the fundamental frequency. Thus, there would be a larger separation in the index values between the two cases for the first pulsation index than for the second pulsation index. Therefore, if the task is to discriminate the flow case from the no flow case, the first pulsation index is preferred over the second pulsation index.

The difference between the first and third pulsation indices only lies in the denominator, i.e., the absence of the second harmonic contribution in the denominator of the third pulsation index. For the no flow case, removing the second harmonic would only remove a small contribution in the denominator leaving the index unaffected. Thus the two indices would have similar values. However, in the flow case, removing the contribution from the second harmonic would lead to a significant reduction in the denominator, and would thus increase the value of the third pulsation index closer to unity than the first pulsation index. Thus, the discrimination between the flow and no flow case would be larger in the case of the third pulsation index. In this heuristic analysis, the third pulsation index is the most preferred among the three indices.

According to one embodiment of the present invention, the pulsation index is computed for several slices, and the maximum among the pulsation index values of all the frequency slices is used to determine whether there is a flow or not. Because the frequency band that best captures the pulsatility information depends on several factors, such as the Doppler frequency, the Doppler angle, and the blood flow conditions (e.g., the condition of the patient's artery, the normal pulsatile flow of the patient, etc.), it is not possible to select a priori the optimal frequency band. Thus, in this embodiment, it is assumed that the maximum pulsation index value would be the most optimal band for finding whether a pulse is present. However, in other embodiments of the present invention, the pulsation index values among the various frequency slices can be manipulated differently in order to determine whether a flow is present.

The bottom graph of FIG. 5 shows the third pulsation index calculated from the data in FIG. 2 (which is reproduced in the top three graphs of FIG. 5) over a slightly extended period of time (60 seconds in FIG. 5 vs. 40 seconds in FIG. 2). It can be seen that the third pulsation index is high for the normal and recovery states, and low for the VF state. In the bottom graph of FIG. 5, the frequency band being used to calculate the pulsation index changes over time depending on which band has the strongest signal, as discussed in the previous paragraph. The changes in frequency band are represented by the different shades of grey the trace takes in the graph. Specifically, the 1150-1350 Hz band is used from 0 to slightly more than 20 seconds, and from before 33 seconds to 60 seconds (i.e., for most of the normal and recovery periods). During VF, the frequency band being used changes several times between the 225-425 Hz, the 680-850 Hz, the 1650-1850 Hz, and the 1150-1350 Hz bands.

The second proposed indicia for flow behavior is directed to measuring the overall flow, regardless of whether it's pulsatile or steady. It is based on the fact that the overall Doppler signal in a specific frequency band should be high for the flow case and low for the no flow case. This indicia, called the “flow index”, would be equivalent to the actual brightness of the pixels in a Doppler spectrogram shown on the display of a conventional ultrasound system. Since the Doppler signal could vary largely from one patient to another, such a quantity would require appropriate normalization. It is preferable to perform this normalization based on the same patient.

One possible way for accomplishing this is to use the fact that many patients at the time of intervention with an AED would already be in a state of VF, i.e., in a state where there is no flow. Thus, one could use this time period to obtain a Doppler signal value and establish this Doppler measurement as the “definition” of the no flow situation. Subsequently, after defibrillation, one could compare the current Doppler power measurements with the prior no flow situation in order to determine whether there is any flow. In one preferred embodiment of an AED using this flow index, the 90^{th} percentile point of the Doppler power spectrum in a particular frequency band is initially computed (while the patient is presumably in VF) over a window of 5 seconds. This initial “no flow” measurement is then used to normalize all future measurements: this normalized measure is the flow index. As can be seen in this example, the flow index is an indicator of the overall flow and is different in nature from the pulsation index. It should be noted that this quantity should be computed only if the AED determines that the patient at the time of intervention is in a state of VF. Obviously, this measure could be used in determining the presence of a post-defibrillation PEA.

As in the preferred embodiment using the pulsation index, the flow index value for several frequency slices is computed and the maximum among the slices is selected as the flow index. In other embodiments, the flow index of several or all the frequency slices could be used. When there is a flow, the flow index should be significantly larger than unity, whereas for the PEA case the flow index should be closer to unity. The choice of the 90^{th} percentile value is somewhat arbitrary, but the maximum value is very susceptible to noise, and the mean value does not exploit the fact that the flow during systolic phase is higher than the mean flow during a cardiac cycle.

The bottom graph of FIG. 6 shows the flow index calculated from the same data as FIG. 5 (and FIG. 2). Again, the flow index is high for the normal and recovery states, and low for the VF state. As discussed above, the frequency band being used to calculate the flow index changes over time depending on which band has the strongest signal, which is represented by the different shades of grey the trace takes in the graph.

Although a flow behavior monitor according to the present invention could present the indicia of flow behavior on a display screen in the trace form shown in the bottom graph of FIGS. 5 and 6, it should be understood that these graph displays are only an example of one of the variety of forms in which the indicia could be presented, as will be more fully discussed in the next section.

The indicia of flow behavior used in the preferred embodiments (i.e., the pulsation index and the flow index) have many advantages over other measurements used to determine flow behavior. Although a measure such as the mean Doppler frequency shift over the entire Doppler spectrogram has the potential to perform well in determining pulsatility, the fact that, for an AED, the flow conditions (flow velocity, angle of flow, etc.) of the patient are not exactly known means the expected behavior of the mean Doppler frequency shift is also unknown. The indicia for flow behavior directed to pulsatile flow disclosed herein do not suffer from this pitfall, and thusly, appear to be more robust measures for pulse state assessment. However, it is possible for the mean Doppler shift within each frequency slice to be used in accordance with the present invention.

As another example of the advantages of the pulsation index, consider using the periodicity of the cross correlation between the Doppler signal and the ECG signal as a measurement of pulsatile flow. When the patient is in a state of pulseless electrical activity (PEA), such a cross-correlation would still show a significant level of periodicity, although lower than for the normal flow case, because the ECG remains periodic even while the Doppler signal is not. One could simply use the value of the cross correlation as a measure of pulsation index, but this has disadvantages. Because the actual value of the cross correlation would depend on the shape of the ECG signal and the Doppler signal, and since the ECG signal in general could assume a variety of shapes depending on the heart condition of the patient, it would be difficult to a priori predict its expected shape, and set a threshold for determining whether there is good correlation with the Doppler signal or not.

Another advantage of the indicia of flow behavior directed to pulsatile flow according to the preferred embodiments of the present invention is that they rely solely on the Doppler signal, and do not rely on any correlation with other signals (e.g., ECG), and hence can be used in stand-alone pulse detection systems.

While the indicia of flow behavior used in the preferred embodiments (i.e., the pulsation index and the flow index) are useful indicators in their own right, it is also possible that these (and other) indicia could be combined together and used in automatically assessing these and other aspects of flow behavior.

The exemplary pulsatile indices used in the preferred embodiments are based on a search for a sinusoidal type of periodicity. However, because the Doppler signal is not sinusoidally periodic, there are harmonics in the power spectrum, which can affect the value of the pulsation index. To avoid this, the second harmonic was removed from the denominator of the third pulsation index. In future embodiments, a more appropriate type of analysis, such as wavelet analysis, could be used to detect the non-sinusoidal periodicity of the Doppler signal.

A primary advantage of a method and system according to the present invention is the ability to adequately assess the flow of a body fluid, such as blood, of an individual without a priori measurements or knowledge of that particular individual. This is of great use in AEDs or other defibrillation devices which require an inexperienced and/or untrained user to determine whether it is appropriate to defibrillate a patient. The robustness of using frequency slices and indicia of flow behavior according to the present invention make the inventive method and system appropriate for defibrillation systems such as AEDs where the possible variation in placement of the ultrasound sensors, the variation in direction of the flow in relation to the sensors, the wide variety of possible patient body shapes and sizes, the wide variety of different “normal” (i.e., healthy) blood flows, the wide variety of different “normal” (i.e., healthy) blood pressures, etc. make it impossible to have too many a priori assumptions about the measurements.

Having described the novel and inventive ultrasound measurement in general, and having described various embodiments of indicia of flow behavior, an exemplary embodiment of a monitoring system according to the present invention will now be described.

Although one of the more important embodiments of the flow behavior monitor according to the present invention is for an AED, it should be understood that the flow behavior monitor may be used in a number of contexts. For example, a flow behavior monitor may be integrated into a video display monitor such as are typically used in hospitals or clinics, in which case the indicia of flow behavior would be shown alongside other measurement results, such as ECG, EEG, SpO₂, CO₂, blood pressure, etc. Thus, it could be used in emergency room equipment, intensive care unit equipment, clinic or doctor's office equipment, ambulance or any mobile caregiving unit equipment, paramedic equipment, etc.

It should also be noted that using an ultrasound technique for such monitoring is preferable in many situations because it is non-invasive, i.e., there is no need to insert a sensor into the patient's body. However, in situations where devices are already inserted into the patient's body, such as during an operating procedure, the need for a non-invasive flow behavior monitor is decreased.

Furthermore, a flow behavior monitor according to the present invention would be particularly well-suited as a fetal heart monitor because of the capability of the pulsation index to discover a weak pulse.

Moreover, a flow behavior monitor according to the present invention is not limited to human and/or animal care or diagnosis. For example, the flow behavior monitor could be used for the analysis of any fluid mass which could be measured by ultrasound Doppler, including, but not limited to, the analysis of underground fluid deposits or streams, the analysis of pipeline flow and/or dynamics, or the analysis of practically any fluid dynamic system.

A non-invasive carotid artery flow behavior monitor is an exemplary embodiment of the present invention. The flow of the carotid artery is a good measurement of how well the heart is perfising the brain, and is especially useful in emergency situations. A non-invasive carotid artery flow behavior monitor would be particularly useful as part of an AED.

A flow behavior monitor according to the present invention could present information in visual and/or audio format.

In some ultrasound systems, a spectral Doppler trace is displayed on a monitor screen. However, a flow behavior monitor according to the present invention, in which indicia of flow behavior are calculated in a plurality of frequency slices, could identify the optimal frequency slice and display the visual trace for just that frequency slice isolated from the rest. Moreover, the monitor could dynamically change frequency slices over time. Of course, it would also be possible to display the band in the same fashion as the screens on the right- and left-hand sides of FIG. 3.

Furthermore, the measurement of the indicia of flow behavior could be integrated into present visual displays on ultrasound monitors. For example, the indicia measurement could be added to the spectral Doppler trace using a color coding scheme, i.e., the color of the trace of the tracing dot would change over time. For example, the color green could represent a normal, healthy pulse (as determined using the indicia measurement), the color red could represent pulseless activity, and the color orange could represent a possible change in the pulse state or an unusual pattern (either determined heuristically or based on a patient's history).

As another example, the indicia measurement could be added as a separate icon or symbol on the display. Whether the indicia is the pulsation index or the flow index, the measurement could be represented as a bar chart going from 0 (no flow or no pulse) to 1 (flow detected or healthy pulse detected). The representation could be a round circle which is either white or black, or one of several colors, or has a diameter which changes size based on the pulse, etc. There are many possible ways an icon or symbol on a display screen could represent the current measurement of the flow behavior indicia.

In an embodiment such as an AED, the monitor could consist of a simple, solitary light bulb which would inform the untrained caregiver whether any pulse is detected either by turning on (pulse detected) or off (no pulse detected) or by changing color (using a color scheme such as the one discussed above). Three or four lights could be used, where either their label or their color indicates the result of the indicia measurement. The possible permutations of ways in which one or more lights on an AED could display the indicia measurement are limitless (and all would be in accordance with the present invention).

Sound can also be used in accordance with the present invention to represent measurements of the flow behavior indicia. For example, changing the frequency of a continuous beeping could indicate the present state of flow behavior, or an alarm could indicate a sudden change in flow behavior, or different tones may indicate the present state of flow behavior. Once again, the possibilities are endless and all possibilities would be in accordance with the present invention.

In one preferred embodiment of a flow behavior monitor, a visual representation of the current state of flow behavior is combined with the audio output of the Doppler signal. In such a preferred embodiment, a Doppler spectral trace is used with a color coding scheme, as discussed above. In addition to this visual information, an audio signal representing the Doppler signal is output on a speaker. Because this signal is in the audible range, a user can listen and get a sense of the flow behavior without having to look at the display screen showing the Doppler spectral trace. The audio output can also be used to inform the user when there is a change in flow behavior (i.e., when the color of the Doppler spectral trace is changing) so that the user will look at the Doppler spectral trace to see exactly what is happening. This “alarm” capability could also be used to signal unusual patterns or changes in the ECG.

Having described the flow behavior monitor in general, and described various possible embodiments of a flow behavior monitor according to the present invention, an exemplary embodiment of a system according to the present invention will now be described.

The exemplary embodiment of the present invention advantageously provides an ultrasound apparatus for monitoring a patient, thereby providing medical staff with diagnostic information related to mechanical activity of the patient's heart. In this one embodiment, the information is acquired using selective calculations of the power of an echo Doppler signal in a plurality of frequency bands of the signal and represented using an operator interface that includes a visual display and, optionally, an audio output. FIG. 7 depicts a block diagram of an exemplary apparatus 100 which detects and/or measures at least one indicia of flow behavior (e.g., state of perfusion, heart beat rate, and/or the pattern of pulsatile activity of the heart of a patient) according to one embodiment of the present invention. Apparatus 100 may be used as a component of a defibrillating system (discussed in reference to FIG. 9 below), a resuscitation system, a monitor, a detector of weak heart beat (e.g., fetal heart beat), and/or other medical systems.

In one presently preferred embodiment, apparatus 100 comprises an ultrasound unit 101 and an operator interface module 103. Ultrasound unit 101 generally includes an ultrasound module 106 and a data processor 108 comprising an echo signal acquisition module 112 and an analyzer 118 of the Doppler signal.

Ultrasound module 106 comprises at least one ultrasonic transducer 114 (four transducers 114 are shown), an RF generator 102, and supporting systems 138. In one embodiment, transducers 114 together form an array 104 that may be disposed upon an application pad (not shown). The supporting systems 138 comprise control and synchronization circuits of generator 102 and ultrasonic transducers 114. Examples of transducer array systems include commonly assigned U.S. Pat. No. 6,575,914 B2, issued Jun. 10, 2003.

Transducer 114 may comprise a transmitter of ultrasound and a receiver of an echo signal. In this embodiment, generator 102 is generally a source of a continuous wave (CW) radio frequency (RF) signal (e.g., 1-10 MHz). In an alternate embodiment, array 104 may comprise transducers 114 that are capable of operating as a transmitter when RF power is ON, or a receiver when the RF power is OFF. In such an embodiment, generator 102 produces pulsed RF power (PW) having duration of an ON time interval of about 0.2 to 20 microseconds and a duty cycle in a range of about 0.2 to 20%.

In operation, generator 102 activates (i.e., excites) the transmitters of transducers 114 to emit an ultrasound beam 132 that propagates in a portion 124 of the body of a patient beneath transducer array 104. The receivers of transducers 114 collect an acoustic echo signal 130 scattered in a region 128 comprising a large blood vessel 126, convert the echo signal into an electrical signal and transmit, via interface 136, to acquisition module 112. In one exemplary application, blood vessel 126 is a carotid artery of the patient. In an embodiment where ultrasound unit 101 and operator interface module 103 are components in an AED, transducer array would be built with the understanding that untrained personnel using the AED might not place transducer array 104 in the appropriate place. For example, the architecture of transducers 114 within transducer array 104 might provide a good deal of redundancy, or the physical shape of transducer array 104 would be appropriately fitted to the part of the neck for which it is intended.

In one embodiment, data processor 108 creates diagnostic information from the measurements of at least one indicia of flow behavior using calculations of the spectral power of the Doppler signal that are selectively performed in a plurality of frequency bands of the signal. Such diagnostic information may comprise the state of perfusion, heart beat rate, and/or a pattern of pulsatile activity of the heart of a patient. The calculations are generally performed, in a digital form, by analyzer 118 of the data processor upon the Doppler signal that is pre-conditioned and converted into a digital domain using echo acquisition module 112.

It should be noted that, in other embodiments, the analysis and/or calculations may be performed in the analog, rather than the digital, domain, e.g., the Doppler signal analyzer 118 might comprise an analog filter bank, and a correlator, etc., as would be known to one of ordinary skill in the art.

More specifically, the diagnostic information is obtained in data processor 108 using calculations of spectral distribution of the power of the Doppler signal. Generally, data processor 108 may use at least one of spectral analysis, Fourier analysis, correlation analysis, auto-correlation analysis of the Doppler signal, an averaged periodogram estimate, parametric analysis, and/or any other computational techniques appropriate for performing the calculations of spectral distribution of the power of the Doppler signal, as would be known to one skilled in the art. In one exemplary embodiment, such calculations are performed in the frequency bands where, during a cardiac cycle, the power of the Doppler signal has the highest signal-to-noise ratio and/or the greatest variation in signal.

In one embodiment, operator interface module 103 comprises a video display 122 (e.g., a cathode ray tube (CRT) display, a liquid crystal display (LCD), a plasma display, etc.), an audio output 120 (e.g., at least one speaker), and a buffer module 116. Buffer module 116 is coupled, using a digital link 140, to Doppler signal analyzer 118. In operation, buffer module 116 converts output signals of analyzer 118 containing the patient's ultrasound diagnostic information in formats that may be supported by video display 122 and audio output 120.

In a further embodiment, operator interface module 103 may comprise features that facilitate interactive control of data processor 108 by an operator (e.g., Emergency Room doctor, surgeon, cardiologist, paramedic, etc.) of apparatus 100. Illustratively, such interactive control functionality may include operator's requests for correlation of the ultrasound diagnostic data presented on video display 122 with information that may be available from a simultaneously operating electrocardiograph (ECG) 134, an blood pressure monitor 502 (shown in FIG. 9 below), or other medical system (not shown). In a further embodiment (not shown), operator interface module 103 may comprise a plurality of video displays 122 and/or audio outputs 120 that facilitate availability of the pertinent patient's diagnostic information to a group of medical professionals.

It should be noted, however, that the ECG signal generally corresponds to the electrical activity of the heart and that the visual output of the ECG of a beating heart and the heart in the state of pulseless electrical activity (PEA) may have similar patterns. As such, exclusive use of the ECG diagnostics may inadvertently result in masking the lack of mechanical activity (i.e., blood pumping functionality) of the patient's heart.

In a presently preferred exemplary embodiment, video display 112 displays measurements of an indicia of flow behavior, and thereby provides diagnostic information regarding at least one of, for example, a state of blood perfusion, a state of pulse, a heart beat rate, and/or flow and/or pulsatile activity of the heart.

FIG. 8 depicts a specific example of a monitor screen shown on video display 122 in apparatus 100 of FIG. 7. The monitor screen displays the spectral power distribution of the Doppler signal during a cardiac cycle. More specifically, a graph 401 depicts an amplitude (y-axis 404) of the spectral power distribution of the Doppler signal versus frequency (x-axis 402) in a frequency range 4 of the Doppler signal. Herein, graphs 406 and 408 illustratively correspond to systolic and diastolic phases of the cardiac cycle of the patient, respectively, and describe a pattern of pulsatile activity of the patient's heart. This, of course, is only an example of a flow behavior monitor according to the present invention, and many other embodiments are possible (as discussed in the beginning of this section).

FIG. 9 is a block diagram of an exemplary programmable defibrillating system 500 in accordance with one embodiment of the present invention. Illustratively, defibrillating system 500 comprises the ultrasound diagnostic apparatus 100 of FIG. 7, an optional ECG 134, an optional ABP monitor 502, a defibrillating unit 508, and a programmable controller 506 of the defibrillating unit. In operation, controller 506 may be programmed by an operator (e.g., Emergency Room doctor, surgeon, cardiologist, paramedic, etc.) upon reviewing, e.g., the ultrasound diagnostic information relating to mechanical activity of the heart on video display 122 or listening to audio output 120.

In one embodiment, the ultrasound diagnostic information is available on the video display 122 in a graphical form and includes at least one of the state of perfusion, heart beat rate, and/or pattern of pulsatile activity of the patient's heart, as discussed above. Additionally, a portion of the information relating to a pattern (i.e., rhythm) of the patient's heart beat rate may be communicated to the operator using audio output 120. In a further embodiment, video displays of at least two components of defibrillating system 500, such as apparatus 110, ECG 134, and ABP monitor 502, may be implemented as a single (i.e., combined or integrated) video display (not shown).

The Doppler signals of a normally beating heart and the heart having deficient blood pumping functionality have easily recognizable audible patterns that may be electronically transmitted to or monitored from a location that is remote to the operator of interface module 103. In another embodiment, audio output 120 may be used to generate pre-recorded warning signals and/or announcements when a controlled parameter (e.g., heart beat rhythm) reaches or exceeds a predetermined clinical value. In a visual format, the diagnostic information may also be shown on video display 122 using, for example, a color-coding scheme. In a further embodiment, apparatus 100 and/or defibrillating system 500 may comprise a plurality of video displays 122 and/or audio outputs 120 that facilitate. availability of the pertinent patient's diagnostic information to a group of medical professionals.

In one exemplary embodiment, the ultrasound diagnostic information may be obtained using the measurements conducted on the patient's carotid artery using ultrasound module 106 and the selective calculations of the spectral power of a Doppler signal performed by data processor 108, as discussed above in reference to FIG. 7. Additionally, such information may be used in diagnosing the state of blood supply of the patient's brain. In a further embodiment, ECG 134 and apparatus 100 may acquire patient's data simultaneously. In this embodiment, the ultrasound diagnostic information may further be cross-correlated with the ECG data. Such correlation additionally increases accuracy and reliability of interpreting the patient's diagnostic information by the operator of system 500. In yet further embodiment, the ultrasound data and ECG data may further be cross-correlated with the data collected by ABP monitor 502 or, alternatively, other diagnostic tool(s).

In one illustrative application, upon review of the diagnostic information, the operator of system 500 makes a decision whether to defibrillate a patient, selects processing parameters of the defibrillating procedure, and correspondingly configures, manually or via a means of electronic controls 514, programmable controller 506. Controller 506 administers execution of the procedure by defibrillating unit 508 that generally comprises a controlled source 510 of high voltage and application electrodes 512 (two electrodes 512 are shown).

In illustrative embodiments discussed above in reference to FIGS. 7 and 9, many portions of apparatus 100 and system 500 are available in medical ultrasound and defibrillation systems from Koninklijke Philips Electronics N.V. of Eindhoven, Netherlands.

Thus, while there have been shown and described and pointed out fundamental novel features of the present invention as applied to preferred embodiments thereof, it will be understood that various omissions and substitutions and changes in the form and details of the devices described and illustrated, and in their operation, and of the methods described may be made by those skilled in the art without departing from the spirit of the present invention. For example, it is expressly intended that all combinations of those elements and/or method steps which perform substantially the same function in substantially the same way to achieve the same results are within the scope of the invention. Substitutions of elements from one described embodiment to another are also fully intended and contemplated. It is the intention, therefore, to be limited only as indicated by the scope of the claims appended hereto.

1. A monitor for presenting results of non-invasively measuring and/or detecting, using an ultrasound device, flow behavior of a fluid within a subject, comprising:
means for presenting at least one of visual and audio output representing at least one measurement of an indicia of flow behavior of the fluid;
wherein total Doppler power is calculated for each of a plurality of frequency slices as a function of time from an ultrasound signal backscattered from the fluid within the subject;
wherein power spectra are calculated for each of the plural frequency slices from the determined total Doppler power for each of the plural frequency slices;
wherein the indicia of flow behavior is calculated for each frequency slice from at least one of the total Doppler power and the power spectrum for that frequency slice; and
wherein at least one of the calculated values of the indicia of flow behavior is used to produce the at least one measurement of the indicia of flow behavior presented by the means for presenting.

2. The flow behavior monitor of claim 1, wherein flow behavior comprises at least one of a state of blood perfusion, a state of pulse, a heart beat rate, and/or flow and/or pulsatile activity of a colloidal or emulsion solution.

3. The flow behavior monitor of claim 1, wherein the means for presenting comprises a display screen.

4. The flow behavior monitor of claim 3, wherein the display screen comprises a cathode ray tube (CRT) display, a liquid crystal display (LCD), and/or a plasma display.

5. The flow behavior monitor of claim 3, wherein the at least one measurement of the indicia of flow behavior is represented on the display screen by an icon and/or symbol.

6. The flow behavior monitor of claim 3, wherein the at least one measurement of the indicia of flow behavior is represented on the display screen as a bar chart, a numeral, a frequency of a flashing light, a color, a number of icons and/or symbols displayed, and/or a shape of icons and/or symbols displayed.

7. The flow behavior monitor of claim 3, wherein at least one of a Doppler spectral trace and a Doppler spectrogram is shown on the display screen.

8. The flow behavior monitor of claim 7, wherein the at least one measurement of the indicia of flow behavior is represented by changing the color of the at least one of a Doppler spectral trace and a Doppler spectrogram.

9. The flow behavior monitor of claim 7, wherein the at least one measurement of the indicia of flow behavior is represented by changing a level of the Doppler spectral trace.

10. The flow behavior monitor of claim 3, wherein other measurements are also shown on the display screen.

11. The flow behavior monitor of claim 10, wherein said other measurements comprise physiological parameter measurements.

12. The flow behavior monitor of claim 10, wherein said other measurements comprise a Doppler spectrogram, a Doppler spectral trace, an ECG (Electrocardiogram), an EEG (Electroencephalogram), SpO₂ (pulse oximetry), CO₂, and/or blood pressure.

13. The flow behavior monitor of claim 1, wherein the means for presenting comprises a speaker for audio output.

14. The flow behavior monitor of claim 1, wherein the means for presenting comprises at least one indicator.

15. The flow behavior monitor of claim 14, wherein the at least one indicator comprises at least one light.

16. The flow behavior monitor of claim 15, wherein the indicia of flow behavior is represented by a color of the at least one light, a number of the at least one light turned on, and/or a frequency of flashing of the at least one light.

17. The flow behavior monitor of claim 1, wherein the flow behavior monitor is connected, either directly or indirectly, to a defibrillator.

18. The flow behavior monitor of claim 17, wherein the defibrillator is an Automated or Semi-Automated External Defibrillator (AED).

19. The flow behavior monitor of claim 1, wherein the subject is a human, an animal, another animate object, and/or an inanimate object.

20. The flow behavior monitor of claim 1, wherein the indicia of flow behavior is the indicia of flow behavior having the highest value among the frequency slices.

21. The flow behavior monitor of claim 1, wherein the indicia of flow behavior comprises a pulsation index, said pulsation index comprising a ratio involving at least one of one or more peaks in the power spectra of a frequency slice and the total power in the power spectra of the frequency slice.

22. The flow behavior monitor of claim 21, wherein the means for presenting at least one of visual and audio output presents a determination of whether there is a pulsatile flow of the fluid within the subject, wherein said determination is made by comparing each of the calculated pulsation indices to a predetermined threshold value, and wherein there is a pulsatile flow if any of the calculated pulsation indices exceeds the predetermined threshold value.

23. The flow behavior monitor of claim 1, wherein the indicia of flow behavior comprises a flow index, said flow index being a later value for a measurement of flow behavior in at least one frequency slice normalized by an initial value for the measurement of flow behavior in the at least one frequency slice.

24. The flow behavior monitor of claim 23, the initial value is obtained while the subject is in ventricular fibrillation, and the later value is obtained after the subject has been defibrillated.

25. The flow behavior monitor of claim 24, wherein the ventricular fibrillation occurred any time from a fraction of a second to a few days before the later value was measured.

26. The flow behavior monitor of claim 23, wherein the means for presenting at least one of visual and audio output presents a determination of whether there is a flow of the fluid within the subject, wherein said determination is made by comparing each of the calculated flow indices to a predetermined threshold value, and wherein there is a flow if any of the calculated flow indices exceeds the predetermined threshold value.

27. The flow behavior monitor of claim 1, wherein the indicia of flow behavior comprises an instantaneous measurement of Doppler power in at least one frequency slice.

28. The flow behavior monitor of claim 1, wherein the means for presenting at least one of visual and audio output presents a warning when the indicia of flow behavior exhibits an unusual pattern and/or crosses a predetermined threshold.

29. The flow behavior monitor of claim 1, wherein the indicia of flow behavior comprises a cross-correlation of the determined power spectra with data collected by an electrocardiograph and/or a blood pressure monitor.

30. The flow behavior monitor of claim 1, wherein a storage device stores data comprising said indicia of flow behavior.

31. A system for presenting results of non-invasively measuring a flow behavior of a fluid within a subject using an ultrasound device, comprising:
processing means operative for:
determining a total Doppler power for each of a plurality of frequency slices as a function of time, wherein said total Doppler power is calculated from an ultrasound signal backscattered from the fluid within the subject;
determining power spectra from the determined total Doppler power whereby each of the plural frequency slices has a power spectrum over the frequencies within that frequency slice; and
calculating a first indicia of flow behavior of the fluid within the subject for each frequency slice;
visual output means for presenting a Doppler spectral trace, wherein the current measurement of the first indicia of flow behavior is represented by a color of the Doppler spectral trace; and
audio output means for output of a second indicia of flow behavior, wherein a user of the system can listen to flow behavior of the fluid within the subject.

32. The system of claim 31, wherein flow behavior comprises at least one of blood perfusion, the pulse state, a heart beat rate, and/or flow and/or pulsatile activity of a colloidal or emulsion solution.

33. The system of claim 31, wherein said processing means comprises at least one of hardware, software, and firmware.

34. The system of claim 31, wherein the processing means cross-correlates one of the determined power spectra or the determined Doppler power with data collected by an electrocardiograph and/or a blood pressure monitor in order to calculate the first indicia of flow behavior.

35. The system of claim 31, wherein the second indicia of flow behavior comprises a Doppler echo signal and/or a heart beat rate.

36. The system of claim 31, wherein said system further comprises: a storage device for storing data comprising at least one of the first and second indicia of flow behavior.

37. The system of claim 31, wherein said system further comprises: a defibrillator.

38. The system of claim 36, wherein the defibrillator is an Automated or Semi-Automated External Defibrillator (AED).
